(19) United States
(12) Patent Application Publication
(10) Pub. No.: US 2021/0228570 A1
(43) Pub. Date: Jul. 29, 2021

(54) COMPOSITIONS AND METHODS FOR OPIOID OVERDOSE RESCUE

(71) Applicant: Purdue Pharma L.P., Stamford, CT (US)

(72) Inventor: John J. Renger, Westport, CT (US)

(21) Appl. No.: 17/059,761

(22) PCT Filed: May 29, 2019
(86) PCT No.: PCT/US19/34309

§ 371 Date: Nov. 30, 2020

Related U.S. Application Data
(60) Provisional application No. 62/679,112, filed on Jun. 1, 2018.

(51) Int. Cl. A61K 31/485; A61K 31/4515

(52) U.S. Cl. CPC A61K 31/485; A61K 31/4515

(57) ABSTRACT

Disclosed herein are various drug delivery systems, pharmaceutical compositions, dosage forms, and kits for providing opioid overdose rescue to a patient as well as a method for providing opioid overdose rescue to a patient. The drug delivery systems, pharmaceutical compositions, dosage forms, and kits may comprise an opioid antagonist and an antipsychotic agent.

### FIELD OF THE INVENTION

The present invention relates to the field of pharmaceutical compositions for rescuing a patient from an opioid overdose, methods of providing opioid overdose rescue, drug delivery systems and opioid overdose rescue kits.

### BACKGROUND OF THE INVENTION

Pharmaceutical products are sometimes subject to abuse. For example, a particular dose of opioid analgesic may be more potent when administered parenterally as compared to the same dose administered orally. Abusing a pharmaceutical product may result in an overdose that could potentially be fatal. To counteract overdose effects, emergency personnel may administer an antidote. When the effects of the overdose are reversed, a patient may become distraught and confused as to their location and/or as to the presence of emergency personnel around them. As a result, a patient may experience a manic behavior that could be harmful to their personal safety and/or to the safety of individuals in their vicinity. In certain circumstances, to prevent harm to the patient and individuals in the vicinity, emergency personnel may attempt to physically restrain the patient upon recovery. In other circumstances, emergency personnel may place the patient in handcuffs such that the patient will be restrained upon recovery. Both of these options can exacerbate the confusion of the patient and the resultant manic behavior instead of reducing harm to the patient or other individuals.

There exists a need in the art for an overdose rescue kit, a pharmaceutical composition for rescuing a patient from overdose, and/or a method of rescuing a patient from an overdose that could counteract the effects of overdose while also providing a physiological and/or chemical restraint that will inhibit a patient's manic behavior.

### OBJECTS AND SUMMARY OF THE INVENTION

It is an object of certain embodiments of the present invention to provide a method for providing overdose (e.g., opioid overdose) rescue to a patient while counteracting manic behavior.

It is an object of certain embodiments of the present invention to provide a drug delivery system for rescuing a patient from overdose (e.g., opioid overdose) while counteracting manic behavior.

It is an object of certain embodiments of the present invention to provide a pharmaceutical composition for rescuing a patient from overdose (e.g., opioid overdose) while counteracting manic behavior.

It is an object of certain embodiments of the present invention to provide an overdose (e.g., opioid overdose) rescue kit for rescuing a patient from overdose while counteracting manic behavior.

The above objects of the present invention and others may be achieved by the present invention which in certain embodiments is directed to a method of providing opioid overdose rescue to a patient. The method may comprise administering to a patient in need thereof an opioid antagonist and an antipsychotic agent. The opioid antagonist and antipsychotic agent may be administered by the same route or by different routes. The administration routes of the opioid antagonist and the antipsychotic agents may be independently selected from the group consisting of parenteral administration (e.g., intramuscular, intraperitoneal, intravenous, and subcutaneous), nasal administration, and inhalation. In some embodiments, the opioid antagonist and antipsychotic agents may be administered concurrently, simultaneously, sequentially, or in any other order. In some embodiments, the method may further comprise identifying that the patient is experiencing an opioid overdose.

In some embodiments, the present invention is directed to a drug delivery system. The drug delivery system may comprise at least one container, an effective amount of an opioid antagonist to counteract an opioid overdose, and an effective amount of an antipsychotic agent to counteract a manic behavior. In certain embodiments, the opioid antagonist and the antipsychotic agent may be held in the same container. In other embodiments, the opioid antagonist and the antipsychotic agent may be held in different containers.

In some embodiments, the present invention is directed to a pharmaceutical composition. The pharmaceutical composition may comprise an effective amount of an opioid antagonist to counteract an opioid overdose and an effective amount of an antipsychotic agent to counteract a manic behavior that may be triggered by the administration of the opioid antagonist and the sudden awakening from overdose to unfamiliar surroundings, possibly restrained in handcuffs or to a hospital bed, and possibly in the presence of rescue personnel (such as first responders including ambulance operators, nurses, doctors, police officers, fire fighters etc.). In one embodiment, the pharmaceutical composition may comprise a combination of the opioid antagonist and the antipsychotic agent. The pharmaceutical composition may be formulated as a solution, a dispersion, a suspension, a powder to be suspended, an injection, an aerosol, or an inhalant.

In some embodiments, the present invention is directed to a dosage form comprising a combination of an opioid antagonist and an antipsychotic agent, wherein the opioid antagonist and the antipsychotic agent are the sole active agents in the dosage form and other components may be present as inactive ingredients.

In some embodiments, the present invention is directed to a rescue kit. The rescue kit may comprise a parenteral delivery system. The parenteral delivery system may comprise an effective amount of an opioid antagonist to counteract an opioid overdose and an effective amount of an antipsychotic agent to counteract manic behavior triggered by the administration of the opioid antagonist and the sudden awakening from overdose to unfamiliar surroundings, possibly restrained in handcuffs or to a hospital bed, and possibly in the presence of rescue personnel (such as first responders including ambulance operators, nurses, doctors, police officers, fire fighters etc.). The opioid antagonist and the antipsychotic agent may be formulated for parenteral administration.

In some embodiments, the rescue kit may comprise a nasal delivery system. The nasal delivery system may comprise an opioid antagonist formulated for nasal administration and an antipsychotic agent formulated for nasal administration. The opioid antagonist may be present in an effective amount to counteract opioid overdose. The antipsychotic agent may be present in an effective amount to counteract manic behavior.

In some embodiments, the rescue kit may comprise a nasal delivery system comprising an effective amount of an opioid antagonist to counteract an opioid overdose and a parenteral delivery system comprising an effective amount of an antipsychotic agent to counteract manic behavior. The opioid antagonist may be formulated for nasal administration. The antipsychotic agent may be formulated for parenteral administration.

In some embodiments, the rescue kit may comprise a parenteral delivery system comprising an effective amount of an opioid antagonist to counteract an opioid overdose and a nasal delivery system comprising an effective amount of an antipsychotic agent to counteract manic behavior. The opioid antagonist may be formulated for parenteral administration. The antipsychotic agent may be formulated for nasal administration.

The opioid antagonist in the various embodiments described herein may be, without limitations, selected from the group consisting of naloxone, naltrexone, and nalmefene, pharmaceutically acceptable salts thereof, pharmaceutically acceptable prodrugs thereof, active metabolites thereof, and combinations thereof. The antipsychotic agent in the various embodiments described herein may be, without limitations, haloperidol or a pharmaceutically acceptable salt thereof.

### Definitions

As used herein, the singular forms “a,” “an,” and “the” include plural references unless the context clearly indicates otherwise. Thus, for example, reference to “an opioid antagonist” includes a single opioid antagonist as well as a mixture of two or more different opioid antagonists; reference to an “antipsychotic agent” includes a single antipsychotic agent as well as a mixture of two or more different antipsychotic agents; and reference to an “excipient” includes a single excipient as well as a mixture of two or more different excipients, and the like.

As used herein, the term “about” in connection with a measured quantity, refers to the normal variations in that measured quantity, as expected by one of ordinary skill in the art in making the measurement and exercising a level of care commensurate with the objective of measurement and the precision of the measuring equipment. In certain embodiments, the term “about” includes the recited number ±10%, such that “about 10” would include from 9 to 11.

As used herein, the terms “active agent” refer to any material that is intended to produce a therapeutic, prophylactic, or other intended effect, whether or not approved by a government agency for that purpose. These terms with respect to specific agents include all pharmaceutically active agents, all pharmaceutically acceptable salts thereof, stereoisomers, crystalline forms, co-crystals, ether, esters, hydrates, solvates, and mixtures thereof, where the form is pharmaceutically active.

As used herein, the terms “therapeutically effective” or an “effective amount” refers to the amount of drug or the rate of drug administration needed to produce a desired therapeutic result.

As used herein, the term “stereoisomers” is a general term for all isomers of individual molecules that differ only in the orientation of their atoms in space. It includes enantiomers and isomers of compounds with one or more chiral centers that are not mirror images of one another (diastereomers).

The term “enantiomer” or “enantiomeric” refers to a molecule that is non-superimposable on its minor image and hence optically active wherein the enantiomer rotates the plane of polarized light in one direction by a certain degree, and its minor image rotates the plane of polarized light by the same degree but in the opposite direction.

The term “chiral center” refers to a carbon atom to which four different groups are attached.

The term “racemic” refers to a mixture of enantiomers.

The term “resolution” refers to the separation or concentration or depletion of one of the two enantiomeric forms of a molecule.

The term “patient” refers to a subject, particularly a human, who has presented a clinical manifestation of a particular symptom or symptoms suggesting the need for treatment, who is treated prophylactically for a condition, or who has been diagnosed with a condition to be treated. The term “subject” encompasses the definition of the term “patient” and does not exclude individuals who are otherwise healthy.

The terms “treatment of” and “treating” include the administration of an active agent(s) with the intent to lessen the severity of or prevent a condition, e.g., manic behavior.

The terms “prevention of” and “preventing” include the avoidance of the onset of a condition, e.g., manic behavior.

The term “condition” or “conditions” refers to those medical conditions, such as manic behavior, that can be treated, mitigated or prevented by administration to a subject of an effective amount of an active agent (such as an antipsychotic agent or pharmaceutically acceptable salt thereof).

The term “manic behavior” refers to a medical condition that may be characterized through physical and mental manifestations that may be expressed by one or more of the following symptoms: irritability, anxiety, aggressiveness, violence to self and others, hypersensitivity, hyper vigilance, impulsivity, a compulsion to over explain, sudden increase in energy levels, decreased need for sleep, hyperactivity, disorientation, speech related issues, incoherence, increase in risky behavior, inattentiveness, delusions, inflated self-esteem, grandiosity, distractibility, etc.

The term “combative behavior” refers to a subject's manifestation of violent, irritable, and/or aggressive symptoms that could results in physical or mental harm to the subject and/or to his surroundings and/or to the person administering the antagonist, which may include subjects as well as objects.

The term “extended release” refers to an active agent that is released over a period of time, e.g., to provide a once daily or twice daily dosage form.

The terms “delayed release” refer to an active agent being released after the occurrence of an event. The event may be the passage of time, a trigger such as change in pH, or any other comparable event as understood by one of ordinary skill in the art.

The term “immediate release” refers to the release of at least about 80%, at least about 85%, at least about 90%, at least about 95%, or at least about 98% of an active agent in a time from about 5 minutes, about 15 minutes, about 30 minutes, about 45 minutes or about 60 minutes after administration to a subject. In the case of oral dosage forms, the release can be measured by in-vitro dissolution in a USP Apparatus 1 (#40 mesh basket) in 900 ml 0.1N HCl at room temperature. In certain embodiments, immediate release can be defined as the onset of a therapeutic effect after administration, e.g., within about 30 minutes, within about 15 minutes, within about 10 minutes, or within about 5 minutes.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to illuminate certain materials and methods and does not pose a limitation on scope. No language in the specification should be construed as indicating any non-claimed element as essential to the practice of the disclosed materials and methods.

### DETAILED DESCRIPTION

### Dosage Forms and Pharmaceutical Compositions

According to various embodiments, the present disclosure is related to a pharmaceutical composition. The pharmaceutical composition may comprise an effective amount of an opioid antagonist to counteract an opioid overdose. The pharmaceutical composition further comprises an effective amount of an antipsychotic agent to counteract manic behavior that may be triggered by the administration of the opioid antagonist and the sudden awakening from overdose to unfamiliar surroundings, possibly restrained in handcuffs or to a hospital bed, and possibly in the presence of rescue personnel (such as first responders including ambulance operators, nurses, doctors, police officers, fire fighters etc.). Upon administration of the pharmaceutical composition (e.g., by nasal administration), a therapeutically effective amount of the antipsychotic agent will be bioavailable upon opioid rescue or within a short time (e.g., less than about 5 minutes, less than about 3 minutes or less than about 1 minute) after opioid rescue. In this manner, when a subject wakes up after being rescued, the effect of the antipsychotic agent may inhibit any combative behavior that the subject would otherwise manifest.

In one embodiment, the pharmaceutical composition may comprise a combination of the opioid antagonist and the antipsychotic agent.

In one embodiment, the present disclosure is related to a dosage form comprising an active agent combination, with the combination consisting of an opioid antagonist and an antipsychotic agent (e.g., haloperidol or a pharmaceutically acceptable salt thereof).

In one embodiment, the present disclosure is related to a dosage form consisting of, or consisting essentially of, an opioid antagonist, an antipsychotic agent (e.g., haloperidol or a pharmaceutically acceptable salt thereof), and one or more pharmaceutically acceptable excipients.

In some embodiments, the pharmaceutical composition may be formulated as a solution, as a dispersion, as a suspension, as an injection, as a powder, as granules, as an aerosol, or as an inhalant. In one embodiment, the pharmaceutical composition may be formulated as a solution. In one embodiment, the pharmaceutical composition may be formulated as an injection, e.g. an intramuscular, subcutaneous, or an intravenous injection. In one embodiment, the pharmaceutical composition may be formulated as an intramuscular injection. In one embodiment, the pharmaceutical composition may be formulated as a powder or as a granule that may be dissolved in a solvent rendering the composition suitable, e.g., for parenteral or intranasal administration. In one embodiment, the pharmaceutical composition may be formulated as an aerosol for inhalation. In one embodiment, the pharmaceutical composition may be formulated as an inhalant.

In some embodiments, the manic behavior comprises a physically or a mentally combative behavior by the subject.

### Release Rates

The release profile of the opioid antagonist and/or of the antipsychotic agent may be, independently, an immediate release profile, an extended release profile, a delayed release profile etc. For instance, in certain embodiments, the opioid antagonist and the antipsychotic agent may both have an immediate release profile. In other embodiments, the antipsychotic agent may have an extended release profile. In yet other embodiments, the antipsychotic agent may have a delayed release profile. In certain embodiments, the release profile of the opioid antagonist may be different from the release profile of the antipsychotic agent. For instance, in one embodiment, the opioid antagonist may have an immediate release profile and the antipsychotic agent may have an extended release profile. In another embodiment, the opioid antagonist may have an immediate release profile and the antipsychotic agent may have a delayed release profile.

### Active Agents

The delivery systems, kits, pharmaceutical compositions, and dosage forms according to the disclosure include various active agents and their pharmaceutically acceptable salts thereof. Pharmaceutically acceptable salts include, but are not limited to, inorganic acid salts such as hydrochloride, hydrobromide, sulfate, phosphate and the like; organic acid salts such as formate, acetate, trifluoroacetate, maleate, tartrate and the like; sulfonates such as methanesulfonate, benzenesulfonate, p-toluenesulfonate, and the like; amino acid salts such as arginate, asparginate, glutamate and the like, and metal salts such as sodium salt, potassium salt, cesium salt and the like; alkaline earth metals such as calcium salt, magnesium salt and the like; organic amine salts such as triethylamine salt, pyridine salt, nicotine salt, ethanolamine salt, triethanolamine salt, dicyclohexylamine salt, N,N′-dibenzylethylenediamine salt and the like.

According to certain embodiments, the delivery systems, kits, pharmaceutical compositions, and dosage forms according to the disclosure include an opioid antagonist. The opioid antagonist may be selected from the group consisting of naloxone, naltrexone, nalmefene, cyclazocine, levallorphan, samidorphan, methylsamidorphan, nalodeine, alvimopan, methylnaltrexone, naloxegol, naloxol, 6β-naltrexol, axelopran, bevenopran, naldemedine, cyprodime, naltrindole, norbinaltorphimine, pharmaceutically acceptable salts thereof, active metabolites thereof, and combinations thereof.

In certain embodiments, the opioid antagonist is selected from the group consisting of naloxone, naltrexone, nalmefene, pharmaceutically acceptable salts thereof, active metabolites thereof, and combinations thereof. In one embodiment, the opioid antagonist comprises naloxone or a pharmaceutically acceptable salt thereof. In one embodiment, the opioid antagonist comprises naltrexone or a pharmaceutically acceptable salt thereof. In one embodiment, the opioid antagonist comprises nalmefene or a pharmaceutically acceptable salt thereof.

In certain embodiments, the opioid antagonist is nalmefene or a pharmaceutically acceptable salt thereof is present at about 0.1 mg/ml to about 5 mg/ml, about 0.3 mg/ml to about 2.5 mg/ml, about 0.5 mg/ml to about 1.5 mg/ml, or about 1 mg/ml and is suitable for parenteral administration. In certain embodiments, the opioid antagonist is nalmefene or a pharmaceutically acceptable salt thereof is present at about 0.4 mg/ml to about 20 mg/ml, about 1.2 mg/ml to about 10 mg/ml, about 1.6 mg/ml to about 8 mg/ml, about 2 mg/ml to about 6 mg/ml, or about 4 mg/ml and is suitable for nasal administration.

In certain embodiments, the opioid antagonist comprises, per dosing unit, an amount of ≥0.5 mg naloxone HCl, an amount of equivalent to between about 0.6 mg naloxone HCl to about 12 mg naloxone HCl, about 0.6 mg naloxone HCl to about 6 mg naloxone HCl, about 0.6 mg naloxone HCl to about 3 mg naloxone HCl, about 0.6 mg naloxone HCl to about 3.75 mg naloxone HCl, about 0.6 mg naloxone HCl to about 2.0 naloxone HCl, about 0.65 mg naloxone HCl to about 20 mg naloxone HCl, about 0.5 mg naloxone HCl to about 15 mg naloxone HCl, about 0.5 mg naloxone HCl to about 10 mg naloxone HCl, about 3 mg naloxone HCl to about 5 mg naloxone HCl, about 0.65 mg naloxone HCl to about 0.8 mg naloxone HCl or about 1.3 mg naloxone HCl to about 1.6 mg naloxone HCl, about 0.6 mg naloxone HCl, about 0.7 mg naloxone HCl, about 0.8 mg naloxone HCl, about 0.9 mg naloxone HCl, about 1.0 mg naloxone HCl, about 1.1 mg naloxone HCl, about 1.2 mg naloxone HCl, about 1.3 mg naloxone HCl, about 1.4 mg naloxone HCl, about 1.5 mg naloxone HCl, about 1.6 mg naloxone HCl, about 1.8 mg naloxone HCl, about 2.0 mg naloxone HCl, or about 2.2 mg naloxone HCl, or an equivalent amount of Naloxone base or a pharmaceutically acceptable salt thereof. The amount of opioid antagonist per unit dose may be dissolved in an application fluid of a volume of about 1000 μl, about 500 μl, about about 250 μl, or about 200 μl. The volume per dosing unit may range from about 35 μl to about 200 μl, from about 50 μl to about 200 μl, from about 100 μl to about 200 μl, from about 100 μl to about 150 μl, from about 35 μl to about 250 μl, from about 75 μl to about 250 μl, from about 75 μl to about 200 μl, about 200 μl, about 150 μl, about 100 μl, about 75 0, or about 50 μl.

According to certain embodiments, the delivery systems, kits, pharmaceutical compositions, and dosage forms according to the disclosure further comprise an antipsychotic agent. In some embodiments, the antipsychotic agent is selected from the group consisting of butyrophenones, diphenylbutylpiperidines, phenothiazines, thioxanthenes, benzamides, tricyclics, benzisoxazoles or benzisothiazoles, phenylpiperazines or quinolinones, blonanserin, pimavanserin, sertindole, molindone, pharmaceutically acceptable salts thereof, active metabolites thereof, and combinations thereof.

In some embodiments, the antipsychotic agent is a butyrophenone. The butyrophenone may be selected from the group consisting of benperidol, bromperidol, droperidol, haloperidol, melperone, pipamperone, timiperone, spiperone, pharmaceutically acceptable salts thereof, active metabolites thereof, and combinations thereof.

In some embodiments, the antipsychotic agent is a diphenylbutylpiperidine. The diphenylbutylpiperidine may be selected from the group consisting of fluspirilene, penfluridol, pimozide, pharmaceutically acceptable salts thereof, active metabolites thereof, and combinations thereof.

In some embodiments, the antipsychotic agent is a phenothiazine. The phenothiazine may be selected from the group consisting of acepromazine, chlorpromazine, cyamemazine, dixyrazine, fluphenazine, levomepromazine, mesoridazine, perazine, periciazine, perphenazine, pipotiazine, prochlorperazine, promazine, promethazine, prothipendyl, thioproperazine, thioridazine, trifluoperazine, triflupromazine, pharmaceutically acceptable salts thereof, active metabolites thereof, and combinations thereof.

In some embodiments, the antipsychotic agent is a thioxanthene. The thioxanthene may be selected from the group consisting of chlorprothixene, clopenthixol, flupentixol, thiothixene, zuclopenthixol, pharmaceutically acceptable salts thereof, active metabolites thereof, and combinations thereof.

In some embodiments, the antipsychotic agent is a benzamide. The benzamide may be selected from the group consisting of sulpiride, sultopride, veralipride, amisulpride, nemonapride, remoxipride, levosulpiride, tiapride, pharmaceutically acceptable salts thereof, active metabolites thereof, and combinations thereof.

In some embodiments, the antipsychotic agent may comprise a tricyclic. The tricyclic may be selected from the group consisting of carpipramine, clocapramine, clorotepine, clotiapine, loxapine, mosapramine, asenapine, clozapine, olanzapine, quetiapine, zotepine, pharmaceutically acceptable salts thereof, active metabolites thereof, and combinations thereof.

In some embodiments, the antipsychotic agent is a benzisoxazole or benzisothiazole. The benzisoxazole or benzisothiazole may be selected from the group consisting of iloperidone, lurasidone, paliperidone, paliperidone palmitate, perospirone, risperidone, ziprasidone, pharmaceutically acceptable salts thereof, active metabolites thereof, and combinations thereof.

In some embodiments, the antipsychotic agent is a phenylpiperazine or a quinolinone. The phenylpiperazine or quinolinone may be selected from the group consisting of aripiprazole, aripiprazole lauroxil, brexpiprazole, cariprazine, pharmaceutically acceptable salts thereof, active metabolites thereof, and combinations thereof.

In one embodiment, the antipsychotic agent is haloperidol or a pharmaceutically acceptable salt thereof. In one embodiment, the opioid antagonist is naloxone or a pharmaceutically acceptable salt thereof and the antipsychotic agent is haloperidol or a pharmaceutically acceptable salt thereof. In another embodiment, the opioid antagonist is naltrexone or a pharmaceutically acceptable salt thereof and the antipsychotic agent is haloperidol or a pharmaceutically acceptable salt thereof. In a further embodiment, the opioid antagonist is nalmefene or a pharmaceutically acceptable salt thereof and the antipsychotic agent is haloperidol or a pharmaceutically acceptable salt thereof.

In certain embodiments, the antipsychotic agent, per unit dose, comprises about 2 mg to about 40 mg, about 2 mg to about 20 mg, about 5 mg to about 15 mg, about 2 mg to about 10 mg, about 10 mg to about 20 mg, about 5 mg to about 10 mg, about 10 mg to about 15 mg, about 15 mg to about 20 mg, or about 7 mg to about 12 mg haloperidol or a pharmaceutically acceptable salt thereof suitable for parenteral administration. Haloperidol or a pharmaceutically acceptable salt thereof suitable for intranasal administration or for inhalation may comprise dosages, per unit dose, that are typically 3-4 times greater than the dosages, per unit dose, of haloperidol or a pharmaceutically acceptable salt thereof suitable for parenteral administration. For instance, the antipsychotic agent, per unit dose, may comprise about 8 mg to about 160 mg, about 8 mg to about 80 mg, about 20 mg to about 60 mg, about 8 mg to about 40 mg, about 40 mg to about 80 mg, about 20 mg to about 40 mg, about 40 mg to about 60 mg, about 60 mg to about 80 mg, or about 28 mg to about 48 mg haloperidol or a pharmaceutically acceptable salt thereof suitable for intranasal administration.

In certain embodiments, the delivery systems, kits, pharmaceutical compositions, and dosage forms discussed herein may further comprise and active agent selected from the group consisting of tranquilizers, CNS depressants, CNS stimulants, sedative hypnotics, and mixtures thereof.

In certain embodiments, the pharmaceutical composition and dosage forms disclosed herein may comprise from about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, or about 7% to about 8%, about 9%, about 10%, about 11%, about 12%, about 13%, about 14%, about 15%, about 16%, about 17%, about 18%, about 19%, about 20%, about 25%, about 30%, about 35%, about 40%, about 45%, about 50%, about 60%, about 70%, or about 80% an active agent (e.g., opioid antagonist and/or antipsychotic agent) per dosage form. In certain embodiments, the pharmaceutical composition and dosage forms disclosed herein may comprise from about 0.1% to about 80%, from about 0.5% to about 30%, or from about 1% to about 10% of an active agent (e.g., opioid antagonist and/or antipsychotic agent) per dosage form. The concentrations of active agent per dosage form may be expressed in (w/w) or in (w/v).

In certain embodiments, the weight ratio of the opioid antagonist to the antipsychotic agent, per unit dose, ranges from about 1000:1 to about 1:1000, from about 500:1 to about 1:500, from about 300:1 to about 1:300, from about 200:1 to about 1:200, from about 100:1 to about 1:100, from about 80:1 to about 1:80, from about 50:1 to about 1:50, from about 40:1 to about 1:40, from about 30:1 to about 1:30, from about 20:1 to about 1:20, from about 10:1 to about 1:10, from about 5:1 to about 1:5, from about 4:1 to about 1:4, from about 3:1 to about 1:3, from about 2:1 to about 1:2, from about 1:1 to about 1:2, from about 1:1 to about 1:3, from about 1:1 to about 1:4, from about 1:1 to about 1:5, from about 1:1 to about 1:10, from about 1:1 to about 1:20, from about 1:1 to about 1:30, from about 1:1 to about 1:40, from about 1:1 to about 1:50, from about 1:1 to about 1:80, from about 1:1 to about 1:100, from about 1:1 to about 1:200, from about 1:1 to about 1:300, from about 1:1 to about 1:500, from about 1:1 to about 1:1000, from about 2:1 to about 1:1, from about 3:1 to about 1:1, from about 4:1 to about 1:1, from about 5:1 to about 1:1, from about 10:1 to about 1:1, from about 20:1 to about 1:1, from about 30:1 to about 1:1, from about 40:1 to about 1:1, from about 50:1 to about 1:1, from about 80:1 to about 1:1, from about 100:1 to about 1:1, from about 200:1 to about 1:1, from about 300:1 to about 1:1, from about 500:1 to about 1:1, or from about 1000:1 to about 1:1.

In certain embodiments, the pharmaceutical compositions and dosage forms disclosed herein comprise a greater weight amount of antipsychotic agent than opioid antagonist. In other embodiments, the pharmaceutical compositions and dosage forms disclosed herein comprise a greater weight amount of opioid antagonist than antipsychotic agent. In an embodiment, the weight amount of antipsychotic agent may be the same as the weight amount of the opioid antagonist.

The amount of active agent (antipsychotic agent or opioid antagonist) may depend, in part, on the intended method of administration. For instance, a greater amount of active agent may be included in a pharmaceutical composition intended for nasal administration than in a pharmaceutical composition intended for parenteral administration due to the reduced bioavailability of active agents delivered via the nasal route.

### Pharmaceutically Acceptable Excipients

The delivery systems, kits, pharmaceutical compositions, and dosage forms according to the disclosure may comprise one or more pharmaceutically acceptable carriers and excipients. Examples of possible pharmaceutically acceptable carriers and excipients are described in the Handbook of Pharmaceutical Excipients, American Pharmaceutical Association (6^{th} Edition, 2009 Publication), which is incorporated by reference herein. Carriers and excipients suitable for dosage forms delivered parenterally, nasally, or through inhalation include, but are not limited to, antioxidants, buffering agents, binding agents, carriers, diluents, surfactants, solubilizers, stabilizers, hydrophilic polymers, hydrophobic polymers, absorption or permeability enhancers, preservative, osmotic agents, isotonicity agents, pH adjusting agents, solvents, co-solvents, viscosity agents, gelling agents, suspending agents or combinations thereof.

Suitable excipients in the delivery systems, kits, pharmaceutical compositions, and dosage forms according to the disclosure include, but are not limited to, lactose USP, lactose USP (anhydrous), lactose USP (spray dried), starch USP, directly compressible starch, mannitol USP, sorbitol, dextrose monohydrate, microcrystalline cellulose NF, dibasic calcium phosphate dihydrate NF, sucrose-based diluents, confectioner's sugar, monobasic calcium sulfate monohydrate, calcium sulfate dihydrate NF, calcium lactate trihydrate granular NF, dextrates NF (e.g., Emdex™), dextrose (e.g., Cerelose™), inositol, hydrolyzed cereal solids such as the Maltrons™ and Mor-Rex™, amylose, powdered cellulose (e.g., Elcema™), calcium carbonate, glycine, bentonite, polyvinylpyrrolidone, glyceryl behenate (Compritol™ 888), metallic stearates (e.g., magnesium, calcium and sodium stearates), stearic acid, hydrogenated vegetable oils (e.g., Sterotex™), talc, waxes such as beeswax and carnauba wax, benzalkonium chloride, silica, fumed silica, colloidal silica, calcium stearate, long chain fatty alcohols, phenyl ethyl alcohol, boric acid, methylparaben, sodium benzoate and sodium acetate, sodium chloride, DL-leucine, polyethylene glycols (e.g., Carbowax™ 4000 and Carbowax™ 6000), sodium oleate, sodium benzoate, benzoic acid, sodium acetate, sodium lauryl sulfate, sodium stearyl fumarate (Pruv™), magnesium lauryl sulfate, stearic acid, stearyl alcohol, mineral oil, paraffin, micro-crystalline cellulose, glycerin, propylene glycol, and the like and combinations thereof.

Suitable surfactants in the delivery systems, kits, pharmaceutical compositions, and dosage forms according to the disclosure include, but are not limited to Polysorbate 80 NF, polyoxyethylene 20 sorbitan monolaurate, polyoxyethylene (4) sorbitan monolaurate, polyoxyethylene 20 sorbitan monopalmitate, polyoxyethylene 20 sorbitan monostearate, polyoxyethylene (4) sorbitan monostearate, polyoxyethylene 20 sorbitan tristearate, polyoxyethylene (5) sorbitan monooleate, polyoxyethylene 20 sorbitan trioleate, polyoxyethylene 20 sorbitan monoisostearate, sorbitan monooleate, sorbitan monolaurate, sorbitan monopalmitate, sorbitan monostearate, sorbitan trilaurate, sorbitan trioleate, sorbitan tristearate, and the like, and mixtures thereof,

Suitable isotonicity agents in the delivery systems, kits, pharmaceutical compositions, and dosage forms according to the disclosure include, but are not limited to dextrose, lactose, sodium chloride, calcium chloride, magnesium chloride, sorbitol, sucrose, mannitol, trehalose, raffinose, polyethylene glycol, hydroxyethyl starch, glycine, and the like, and mixtures thereof.

Suitable suspending agents in the delivery systems, kits, pharmaceutical compositions, and dosage forms according to the disclosure include, but are not limited to microcrystalline cellulose, carboxymethylcellulose sodium NF, polyacrylic acid, magnesium aluminum silicate, xanthan gum, and the like, and mixtures thereof. In certain embodiments, the pharmaceutical compositions and dosage forms may include one or more suspending agents in an amount of from about 0.1% to about 15%, or from about 0.25% to about 10%, or from about 1% to about 8%, of the total volume of the pharmaceutical composition or dosage form. The concentration of the suspending agent in the pharmaceutical composition or dosage form may be expressed as (w/w) or (w/v).

In some embodiments, the present invention is directed to methods of preparing the dosage forms and pharmaceutical compositions disclosed herein.

In some embodiments, preparation of the dosage form comprises dissolving the antipsychotic agent composition and/or the opioid antagonist composition, which may be provided in a form of a powder or granules, in a pharmaceutically acceptable solvent (e.g., 0.9% NaCl), thereby rendering the dissolved dosage form suitable for parenteral or nasal administration.

In some embodiments, preparation of the dosage form comprises combining the antipsychotic agent with a pharmaceutically acceptable carrier and/or excipient, and separately combining the opioid antagonist with a pharmaceutically acceptable carrier and/or excipient. The antipsychotic agent preparation and the opioid antagonist preparation may then be combined to form a final dosage form. In other embodiments, the antipsychotic agent preparation and the opioid antagonist preparation may remain as separate compositions in a single kit.

In some embodiments, preparation of the dosage form comprises combining the antipsychotic agent, the opioid antagonist, and a pharmaceutically acceptable excipient.

In some embodiments, the pH of the dosage form may range from about 2 to about 7, from about 3 to about 6, from about 3 to about 5, from about 3 to about 4, from about 3.5 to about 6, from about 3 to about 5.5, from about 3 to about 4.5, from about 4 to about 6, from about 4 to about 5, or from about 4.5 to about 6.

### Method of Providing Overdose Rescue

In certain embodiments, the present disclosure is directed to a method of providing overdose rescue, such as opioid overdose rescue to a subject in need thereof. The method comprises administering to a patient in need thereof an opioid antagonist and an antipsychotic agent such that the benefit of the activity of each component (opioid antagonist and antipsychotic agent) is obtained. The present invention anticipates that the combination will be administered urgently to a subject experiencing a medical emergency precipitated by opioid overdose. Thus, the combination may be administered typically by a medical practitioner, emergency medical technician, family member, acquaintance, or bystander upon observing the subject experiencing the symptoms of opioid overdose. In some embodiments, the method may further comprise, before the administering step, identifying that the patient is experiencing an opioid overdose.

In certain embodiments, the opioid antagonist is administered to a subject in an effective amount to counteract opioid overdose. In certain embodiments, the antipsychotic agent is administered to a subject in an effective amount to prevent, reduce, or counteract a manic behavior. The manic behavior may be a physically or mentally combative behavior seen in some subjects.

In certain embodiments, the antipsychotic agent is administered to a subject before the subject wakes up. In this manner, the subject may already experience a therapeutic effect of the antipsychotic agent upon awakening or shortly thereafter, which may prophylactically prevent the patient from engaging in a physically or mentally combative behavior after opioid overdose rescue.

In some embodiments, the opioid antagonist and the antipsychotic agent are administered separately. In other embodiments, the opioid antagonist and the antipsychotic agent are administered together as a combination in a single dosage form.

The opioid antagonist and antipsychotic agent may each be administered via a route of administration independently selected from the group consisting of parenteral administration, nasal administration, and inhalation. Parenteral administration may be selected from the group consisting of intramuscular administration, intraperitoneal administration, intravenous administrations, and subcutaneous administration.

In some embodiments, the opioid antagonist and antipsychotic agent may be administered via the same route of administration. In other embodiments, the opioid antagonist and the antipsychotic agent may be administered via different routes of administration.

In one embodiment, the opioid antagonist and the antipsychotic agent are both administered to a subject in need thereof via intramuscular administration.

In another embodiment, the opioid antagonist and the antipsychotic agent are administered to a subject in need thereof via subcutaneous administration.

In another embodiment, the opioid antagonist and the antipsychotic agent are both administered to a subject in need thereof via nasal administration.

In some embodiments, the opioid antagonist and the antipsychotic agent may be administered concurrently, simultaneously, or sequentially.

The term “concurrently” as used herein means that a dose of one agent is administered prior to the end of the dosing interval of another agent. For example, a dose of an opioid antagonist with a particular dosing interval would be concurrently administered with an antipsychotic agent dose administered within dosing interval of the opioid antagonist administration.

The term “simultaneously” as used herein means that a dose of one agent is administered at the same time as another agent, regardless of whether the agents are administered separately via the same or different routes of administration or in a single pharmaceutical composition or dosage form. For example, a dose of an opioid antagonist may be administered at the same time as a dose of an antipsychotic agent.

The term “sequentially” as used herein means that a dose of one agent is administered first and thereafter a dose of another agent is administered second. For example, a dose of an opioid antagonist may be administered and thereafter a dose of an antipsychotic agent may be administered. The subsequent administration of the other agent may be inside or outside the dosing interval of agent that was administered first.

In one embodiment, the opioid antagonist and the antipsychotic agent may be administered concurrently and via the same route (e.g., intramuscular or via nasal administration). In another embodiment, the opioid antagonist and the antipsychotic agent may be administered concurrently and via different routes.

In one embodiment, the opioid antagonist and the antipsychotic agent may be administered simultaneously and via the same route (e.g., intramuscular or via intranasal administration). In another embodiment, the opioid antagonist and the antipsychotic agent may be administered simultaneously and via different routes.

In one embodiment, the opioid antagonist and the antipsychotic agent may be administered sequentially and via the same route (e.g., intramuscular or via nasal administration). In another embodiment, the opioid antagonist and the antipsychotic agent may be administered sequentially and via different routes.

### Kits

In certain embodiments, the present disclosure is directed to an opioid overdose rescue kit.

In one embodiment, the opioid overdose rescue kit comprises a parenteral delivery system. The parenteral delivery system comprises a therapeutically effective amount of an opioid antagonist to counteract an opioid overdose in a subject. The opioid antagonist is formulated for parenteral administration. The parenteral delivery system further comprises a therapeutically effective amount of an antipsychotic agent to counteract manic behavior that might be triggered by administration of the opioid antagonist alone and/or by the sudden awakening from overdose to unfamiliar surroundings, possibly restrained in handcuffs or to a hospital bed, and possibly in the presence of rescue personnel (such as first responders including ambulance operators, nurses, doctors, police officers, fire fighters etc.). The antipsychotic agent may be formulated for parenteral administration.

In one embodiment, the opioid overdose rescue kit comprises a nasal delivery system. The nasal delivery system comprises a therapeutically effective amount of an opioid antagonist formulated for nasal administration. The nasal delivery system may further comprises a therapeutically effective amount of an antipsychotic agent formulated for nasal administration to counteract manic behavior that might be triggered by administration of the opioid antagonist alone and/or by the sudden awakening from overdose to unfamiliar surroundings, possibly restrained in handcuffs or to a hospital bed, and possibly in the presence of rescue personnel (such as first responders including ambulance operators, nurses, doctors, police officers, fire fighters etc.).

In certain embodiment, the opioid overdose rescue kit may comprise a combination of various delivery systems that are compatible with the formulation of the active agent that is to be administered to the patient. For instance, in one embodiment, the opioid overdose rescue kit comprises a nasal delivery system and a parenteral delivery system. The nasal delivery system may comprise a therapeutically effective amount of an opioid antagonist to counteract an opioid overdose. The opioid antagonist is formulated for nasal administration. The parenteral delivery system may comprise a therapeutically effective amount of an antipsychotic agent to counteract manic behavior that might otherwise be triggered in the subject by administration of the opioid antagonist alone and/or by the sudden awakening from overdose to unfamiliar surroundings, possibly restrained in handcuffs or to a hospital bed, and possibly in the presence of rescue personnel (such as first responders including ambulance operators, nurses, doctors, police officers, fire fighters etc.). In one embodiment, the antipsychotic agent is formulated for parenteral administration.

In certain embodiments, the present disclosure is directed to a drug delivery system. The drug delivery system may further comprise a container. In some embodiments, the opioid antagonist and the antipsychotic agent may be held (e.g., combined) in the at least one container. In other embodiments, the opioid antagonist and the antipsychotic agent may be held in separate containers.

In some embodiments, the opioid antagonist and the antipsychotic agent may be held in the same container. In other embodiments, the opioid antagonist and the antipsychotic agent may be held in separate containers.

The at least one container in the drug delivery systems described herein may be selected, without limitations, from a vial, a pre-filled syringe barrel, an injection pen (e.g., similar to an insulin pen), an autoinjector, a cartridge for a nebulizer, an IV fluid bag, a metered or a non-metered spray (nasal or inhaler), or nasal drops. An exemplary autoinjector may be a spring-loaded syringe that may be used by trained and/or untrained personnel. An autoinjector may be administered into the thigh or buttocks of an individual in need thereof (e.g., an individual experiencing opioid overdose). The autoinjector may have the needle tip shielded prior to injection and may include a safety mechanism preventing inadvertent injection. The autoinjector may allow for adjustable injection depth and may further comprise a visual indicator confirming that the full dose has been delivered.

In some embodiments, kits according to the disclosure may further comprise instructional materials, such as, preparation instructions, administration instructions, or safety precautions. In some embodiments, the kits may further comprise an autoinjector, an injection pen, a nebulizer, a nebulizer mask, a needle, or a combination thereof.

### EXAMPLES

The following prophetic examples are set forth to assist in understanding the invention and should not be construed as specifically limiting the invention described and claimed herein. Such variations of the invention, including the substitution of any or all equivalents now known or later developed, which would be within the purview of those skilled in the art, and changes in formulation or minor changes in therapeutic design, are to be considered to fall within the scope of the invention incorporated herein.

### Example 1

| | | |
|---|---|---|
| | Quantity | Concentration |
| Component | per dose (mg) | (mg/ml) |

| | | | | |
|---|---|---|---|---|
| Nalmefene or a | 4 | mg | 4 | mg/ml |
| pharmaceutically acceptable | | | | |
| salt thereof (e.g., | | | | |
| Nalmefene HCl) | | | | |
| Haloperidol or a | 80 | mg | 80 | mg/ml |
| pharmaceutically acceptable | | | | |
| salt thereof | | | | |

Solutions are prepared in 0.9% (w/v) sodium chloride solution (pH adjusted to about 4 to about 5.6, about 4 to about 5, about 4±0.5). The solution is contained in a device suitable for intranasal administration. The solution may also be contained in a device suitable for parenteral administration.

### Example 2

| | | |
|---|---|---|
| | Quantity | Concentration |
| Component | per dose (mg) | (mg/ml) |

| | | | | |
|---|---|---|---|---|
| Naloxone or a | 2 | mg | 2 | mg/ml |
| pharmaceutically acceptable | | | | |
| salt thereof (e.g., | | | | |
| Naloxone HCl) | | | | |
| Haloperidol or a | 80 | mg | 80 | mg/ml |
| pharmaceutically acceptable | | | | |
| salt thereof | | | | |

Solutions are prepared in 0.9% sodium chloride solution (pH adjusted to about 4 to about 5.6, or about 3.5±0.5). The solution is contained in a device suitable for intranasal administration. The solution may also be contained in a device suitable for parenteral administration.

In the foregoing description, numerous specific details are set forth, such as specific materials, dimensions, processes parameters, etc., to provide a thorough understanding of the present invention. The particular features, structures, materials, or characteristics may be combined in any suitable manner in one or more embodiments. The words “example” or “exemplary” are used herein to mean serving as an example, instance, or illustration. Any aspect or design described herein as “example” or “exemplary” is not necessarily to be construed as preferred or advantageous over other aspects or designs. Rather, use of the words “example” or “exemplary” is simply intended to present concepts in a concrete fashion. As used in this application, the term “or” is intended to mean an inclusive “or” rather than an exclusive “or”. That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. Reference throughout this specification to “an embodiment”, “certain embodiments”, or “one embodiment” means that a particular feature, structure, or characteristic described in connection with the embodiment is included in at least one embodiment. Thus, the appearances of the phrase “an embodiment”, “certain embodiments”, or “one embodiment” in various places throughout this specification are not necessarily all referring to the same embodiment.

The present invention has been described with reference to specific exemplary embodiments thereof. The specification and drawings are, accordingly, to be regarded in an illustrative rather than a restrictive sense. Various modifications of the invention in addition to those shown and described herein will become apparent to those skilled in the art and are intended to fall within the scope of the appended claims.

1. A method of providing opioid overdose rescue to a patient comprising:
administering to a subject in need thereof a therapeutically effective amount of opioid antagonist to counteract an opioid overdose and a therapeutically effective amount of an antipsychotic agent to counteract a manic behavior.

2. The method of claim 1, wherein the opioid antagonist is selected from the group consisting of naloxone, naltrexone, nalmefene, cyclazocine, levallorphan, samidorphan, methylsamidorphan, nalodeine, alvimopan, methylnaltrexone, naloxegol, naloxol, 6β-naltrexol, axelopran, bevenopran, naldemedine, cyprodime, naltrindole, norbinaltorphimine, pharmaceutically acceptable salts thereof, and a combinations thereof.

3. The method of claim 2, wherein the opioid antagonist is selected from the group consisting of naloxone, naltrexone, nalmefene, pharmaceutically acceptable salts thereof, and combinations thereof.

4. The method of claim 3, wherein the opioid antagonist comprises naloxone, or a pharmaceutically acceptable salt thereof.

5. The method of claim 3, wherein the opioid antagonist comprises naltrexone, or a pharmaceutically acceptable salt thereof.

6. The method of claim 3, wherein the opioid antagonist comprises nalmefene, or a pharmaceutically acceptable salt thereof.

7. The method of claim 1, wherein the antipsychotic agent is selected from the group consisting of butyrophenones, diphenylbutylpiperidines, phenothiazines, thioxanthenes, benzamides, tricyclics, benzisoxazoles or benzisothiazoles, phenylpiperazines or quinolinones, blonanserin, pimavanserin, sertindole, molindone, pharmaceutically acceptable salts thereof, and combinations thereof.

8. The method of claim 7, wherein the antipsychotic agent is a butyrophenone selected from the group consisting of benperidol, bromperidol, droperidol, haloperidol, melperone, pipamperone, timiperone, spiperone, pharmaceutically acceptable salts thereof, and combinations thereof.

9. The method of claim 7, wherein the antipsychotic agent is a diphenylbutylpiperidine selected from the group consisting of fluspirilene, penfluridol, pimozide, pharmaceutically acceptable salts thereof, and combinations thereof.

10. The method of claim 7, wherein the antipsychotic agent is a phenothiazine selected from the group consisting of acepromazine, chlorpromazine, cyamemazine, dixyrazine, fluphenazine, levomepromazine, mesoridazine, perazine, periciazine, perphenazine, pipotiazine, prochlorperazine, promazine, promethazine, prothipendyl, thioproperazine, thioridazine, trifluoperazine, triflupromazine, pharmaceutically acceptable salts thereof, and combinations thereof.

11. The method of claim 7, wherein the antipsychotic agent is a thioxanthene selected from the group consisting of chlorprothixene, clopenthixol, flupentixol, thiothixene, zuclopenthixol, pharmaceutically acceptable salts thereof, and combinations thereof.

12. The method of claim 7, wherein the antipsychotic agent is a benzamide selected from the group consisting of sulpiride, sultopride, veralipride, amisulpride, nemonapride, remoxipride, levosulpiride, tiapride, pharmaceutically acceptable salts thereof, and combinations thereof.

13. The method of claim 7, wherein the antipsychotic agent is a tricyclic selected from the group consisting of carpipramine, clocapramine, clorotepine, clotiapine, loxapine, mosapramine, asenapine, clozapine, olanzapine, quetiapine, zotepine, pharmaceutically acceptable salts thereof, and combinations thereof.

14. The method of claim 7, wherein the antipsychotic agent is a benzisoxazole or benzisothiazole selected from the group consisting of iloperidone, lurasidone, paliperidone, paliperidone palmitate, perospirone, risperidone, ziprasidone, pharmaceutically acceptable salts thereof, and combinations thereof.

15. The method of claim 7, wherein the antipsychotic agent is a phenylpiperazine or quinolinone selected from the group consisting of aripiprazole, aripiprazole lauroxil, brexpiprazole, cariprazine, pharmaceutically acceptable salts thereof, and combinations thereof.

16. The method of claim 1, wherein the antipsychotic agent is haloperidol or a pharmaceutically acceptable salt thereof.

17. The method of claim 1, wherein the opioid antagonist is naloxone or a pharmaceutically acceptable salt thereof and the antipsychotic agent is haloperidol or a pharmaceutically acceptable salt thereof.

18. The method of claim 1, wherein the opioid antagonist is nalmefene or a pharmaceutically acceptable salt thereof and the antipsychotic agent is haloperidol or a pharmaceutically acceptable salt thereof.

19. The method of claim 1, wherein the opioid antagonist and the antipsychotic agent are administered to the subject via the same route.

20. The method of claim 1, wherein the opioid antagonist and the antipsychotic agent are administered to the subject via different routes.

21.-102. (canceled)
